(19) United States
(12) Patent Application Publication
(10) Pub. No.: US 2015/0210651 A1
(43) Pub. Date: Jul. 30, 2015

(54) VAPOUR DEPOSITION OF ORGANIC UV ABSORBERS ONTO PLASTIC SUBSTRATES

(71) Applicant: BAYER MATERIALSCIENCE AG, Leverkusen (DE)

(72) Inventors: Timo Kuhlmann, Leichlingen (DE); Rafael Oser, Krefeld (DE)

(21) Appl. No.: 14/421,907

(22) PCT Filed: Aug. 19, 2013
(86) PCT No.: PCT/EP2013/067210

§ 371 Date: Feb. 16, 2015

(30) Foreign Application Priority Data
Aug. 23, 2012 (EP) 12181458.6

(51) Int. Cl. C07D 249/18; B05D 7/02; B05D 1/00

(52) U.S. Cl. CPC C07D 249/18; B05D 1/62; B05D 7/02

(57) ABSTRACT

Method for coating a plastics substrate with a functional layer, wherein (a) in a vacuum chamber, an organic UV absorber which comprises at least one chromophore and at least one reactive side chain is evaporated and (b) is brought into contact with at least one surface of the plastics substrate and excited with a plasma, or (c) is excited with a plasma and then brought into contact with at least one surface of the plastics substrate, whereby a functional layer comprising the UV absorber is formed on the surface of the plastics substrate, the plasma being ignited at a pressure of greater than 10⁻⁵ bar and less than 1.013 bar.

### TECHNICAL FIELD OF THE INVENTION

The invention relates to a method for coating a plastics substrate with a functional layer comprising an organic UV absorber by means of plasma-induced vapour deposition, and to a device for carrying out the method. The invention relates further to a plastics substrate produced by means of the method and to the use thereof.

### BACKGROUND OF THE INVENTION

Plastics mouldings are increasingly being used to produce casings for electronic devices, window profiles, headlamp diffusers, bodywork elements, machine covers, vehicle windows and architectural glazing. Plastics mouldings can be extruded or produced by injection moulding with a low outlay in terms of manufacture. In addition, complex shapes can be produced with a large degree of design freedom from plastics material.

In order to be able to use plastics mouldings in a variety of different ways, it is necessary to improve the relatively soft and chemically not very resistant plastics surfaces in order to make them scratch- and wear-resistant, for example, and to protect them from the effects of the weather, in particular UV light. To that end, for example, layers that have the desired properties can be applied to the plastics mouldings.

A known method for applying inorganic or organic layers is plasma polymerisation or plasma-enhanced or plasma-induced vapour deposition (PECVD), which is described inter alia in “G. Benz: Plasmapolymerisation: Überblick und Anwendung als Korrosions- und Zerkratzungsschutzschichten. VDI-Verlag GmbH Düsseldorf, 1989”.

DE 199 24 108 A1 describes plasma polymer layers on a substrate, in the production of which a UV absorber in liquid form is sprayed in vacuo into the vacuum chamber by means of a spray device. It is mentioned as advantageous that the UV absorber comes into contact with the plasma only on the liquid surface of the drop that is sprayed in and only that portion of the UV absorber is there exposed to interactions with the plasma. The remainder of the drop passes through the plasma regardless and is deposited on the substrate. Disadvantages of this method are on the one hand that the fragments of the UV absorber are no longer present with a functioning chromophore after recombination on the substrate, and on the other hand that the recombined fragments have an undesired inherent colour. It is further described that a layer grows on the substrate from the portion of the non-fragmented UV absorber. An embodiment of DE 199 24 108 A1 comprises the simultaneous spraying of the UV absorber with an inorganic precursor. This variant has the disadvantage that the UV absorber on the surface of the drop is already able to react with fragments of the precursor. A layer deposited in that manner is no longer homogeneous and exhibits haze. According to the present invention, the UV absorber is evaporated and not sprayed on in the form of a liquid.

DE 195 228 65 A1 describes a material having improved absorption in the ultraviolet range, which material was obtained by depositing a special organic compound having the structural unit

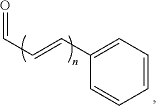

wherein n denotes 0 or 1, on a carrier by the PECVD process. A disadvantage here is that the plastics substrate is not adequately protected from UV radiation. The protection of these layers is inadequate in particular in the range of the spectral sensitivity of the polycarbonate frequently used as the plastics substrate.

DE 199 01 834 A1 describes a method for coating substrates on plastics material, in which the UV absorber, that is to say the layer-forming substance, does not have a reactive side chain. Furthermore, the UV-absorbing substance is evaporated largely in the absence of a plasma, whereas according to the present invention the evaporation takes place with plasma.

FR 2 874 606 A1 describes a method for functionalising transparent layers, in which the functional layer is formed by evaporating a liquid organic substance and distributing it on the substrate, and at the same time a glass-like layer is deposited on the substrate by means of PECVD. The organic substance is selected from the group of the naphthalenes, anthrazenes, pyrenes, anthraquinones and derivatives thereof. A disadvantage of the use of such organic liquids is that uncontrolled decomposition of the organic liquids can occur during exposure in the PECVD process. As a result, some of the desired functions of the organic liquids are no longer available in the resulting layer. The presence in the resulting glass-like layer of fragments of the organic liquids which have not been reproducibly formed can lead to an impairment of the ageing resistance of the layer. Furthermore, the organic liquids chosen in FR 2 874 606 A1 exhibit considerable absorption in the visible wavelength range, which leads to an undesired inherent colour.

WO2004/035667 describes a method for forming UV absorber layers on an inorganic or organic substrate, in which at least one radical-forming initiator is applied together with the UV absorber comprising an ethylenically unsaturated group. Application takes place by spraying in droplet form, whereas in the present method no additional initiator is required and the UV absorber is evaporated.

WO 1999/055471 A1 describes a method for producing a cohesive UV-radiation-absorbing layer on organic or inorganic substrates by means of plasma-enhanced vacuum deposition. In this method, a UV absorber of the hydroxyphenyl-s-triazine class is evaporated in vacuo, exposed to a plasma and thereby deposited on the substrate. Disadvantages of the method described therein are the long process times owing to the low rates of evaporation of the UV absorber. In addition, the effectiveness of the resulting layer as a UV-absorbing layer has not been demonstrated in WO 1999/055471 A1. The mentioned evaluation of the transmission at 380 nm is rather to be regarded as a measure of the discolouration of the layer. Such a discolouration may have occurred, for example, because the process parameters chosen in WO 1999/055471 A1 lead to fragmentation of the UV absorber.

### SUMMARY OF THE INVENTION

The object underlying the invention is to provide a method, which is improved as compared with the prior art, for applying functional layers comprising organic UV absorber to plastics substrates by means of plasma-enhanced vapour deposition.

The object is achieved by a method for coating a plastics substrate with a functional layer, in which
(a) in a vacuum chamber, an organic UV absorber which comprises at least one chromophore and at least one reactive side chain is evaporated and
(b) is brought into contact with at least one surface of the plastics substrate and then excited with a plasma, and/or
(c) is excited with a plasma and then brought into contact with at least one surface of the plastics substrate,
   whereby a functional layer comprising the UV absorber is formed on the surface of the plastics substrate, and wherein the plasma is ignited at a pressure of greater than 10⁻⁵ bar and less than 1.013 bar.

The method according to the invention includes various method embodiments. After evaporation of the UV absorber, the UV absorber can be excited by a plasma and then deposited in the form of a layer on the substrate surface. In this embodiment, the UV absorber passes through the plasma before coming into contact with the substrate surface.

The UV absorber in vapour form can also be excited by the plasma after it has come into contact with the substrate surface and can be deposited in the form of a layer on the substrate surface.

When the plasma is in contact with the plastics substrate, both events can also occur simultaneously, namely the excited UV absorber comes into contact with the substrate, and also UV absorber comes into contact with the substrate and only then is excited.

According to one embodiment of the method, the UV absorber in vapour form can be excited with a plasma at the time of contacting of the substrate surface.

The invention further provides a coated plastics substrate comprising at least one UV-protective layer and optionally at least one scratch-resistant layer, which plastics substrate has been produced by a method according to any one of claims 1 to 14.

Finally, the invention relates to the use of a plastics substrate coated according to any one of claims 1 to 14 as a casing for electronic devices, as a window profile, as a headlamp diffuser, as a bodywork element, as a machine cover, as a vehicle window and as architectural glazing.

### DESCRIPTION OF PREFERRED EMBODIMENTS

By means of the method according to the invention it is possible to produce UV-absorber-comprising functional layers which are clear, colourless and transparent and ensure adequate protection against UV radiation.

Within the meaning of the invention, a “functional layer” is understood as meaning a layer which is so configured or composed that it imparts at least one desired property to the coated substrate. In general, such properties can be improved protection against UV light, IR radiation, higher scratch resistance, hardness or resilience. The UV-absorber-comprising functional layers produced by the method according to the invention are UV-protective layers. In addition, the UV-protective layers produced according to the invention can also have further protective properties, in particular scratch resistance. Functional layers within the meaning of the invention can consequently include layers having UV protection and scratch resistance.

The “clarity” of a functional layer is quantified by the optical parameter for describing the scattering (haze). Clear functional layers within the meaning of the invention have a low haze value. The haze value can be determined, for example, in accordance with ASTM D 1003 using a Haze Gard Plus from Byk-Gardner. Preferably, the haze value of functional layers according to the invention is less than or equal to 5%, particularly preferably less than or equal to 2%, in particular less than or equal to 1%.

A “colourless” layer according to the invention has a low yellowness index. The yellowness index is a value for the yellowing of a material which is inherently colourless and can be determined, for example, in accordance with ASTM D 313 using a Perkin Elmer Lambda 900 spectrophotometer. Preferably, the yellowness index of the functional layers produced by the method according to the invention is less than or equal to 6, particularly preferably less than or equal to 4, in particular less than or equal to 2.5.

A “transparent” layer within the meaning of the invention has a high degree of light transmission, called transmission hereinbelow. The transmission (T_{y}), or extinction, can be determined, for example, using a Perkin Elmer Lambda 900 spectrophotometer in a spectral range from 200 nm to 700 nm of 0°/diffuse in accordance with ISO 13468-1. Preferably, the transmission of the functional layers produced by the method according to the invention on a transparent substrate having a thickness of 3 2 mm is greater than or equal to 85.0%, particularly preferably greater than or equal to 86.0%, in particular greater than or equal to 87.0%.

As a result of the organic UV absorber, the UV-protective layer according to the invention has high UV absorption. A measurement value for the UV absorption is the optical density at 340 nm, referred to as OD340 hereinbelow. This can be determined, for example, using a Perkin Elmer Lambda 900 spectrophotometer. The OD340 is determined from the spectral transmittance T at wavelength 340 nm according to the following formula:

$$OD 340 = {log}_{10} ( \frac{{T}_{sub}}{{T}_{ss}} )$$

where T_{sub} is the transmittance of the uncoated substrate and Tₛₛ is the transmittance of the coated substrate.

Preferably, the OD340 is at least 1.0. Particularly preferably, the OD340 is at least 2.0, and in particular the OD340 is at least 2.15.

The “scratch resistance” of a coated sample can preferably be determined by the Taber Abrasion Test according to DIN 52347 using fourth-generation CS10F wheels with an applied weight of 500 g per wheel, with associated measurement of the increase in the scattered light in accordance with ASTM D 1003 using a Haze Gard Plus from Byk-Gardner. Calculation of the difference value of the haze in % before and after exposure to the friction wheels gives an indication of the quality of the scratch resistance. Coating layers produced by the method according to the invention preferably exhibit a difference in the haze values after exposure to 500 revolutions of less than 20%, particularly preferably a difference of less than 10% and most particularly preferably a difference of less than 5%.

UV Absorber

A UV absorber according to the invention comprises at least one chromophore and at least one reactive side chain. The chromophore is the minimum component necessary for the function of the UV absorber, namely the absorption of ultraviolet radiation. A chromophore selectively absorbs specific frequency ranges of light. The chromophores used according to the invention in particular absorb radiation in the ultraviolet range of the frequency spectrum of light. In principle, any substance which absorbs radiation in the UV spectrum can be used as the chromophore.

In addition, the UV absorber used according to the invention has one or more reactive side chains, which can be the same or different. A reactive side chain desired according to the invention is suitable for protecting the chromophore from fragmentation in the plasma. This is effected, for example, by reaction of the reactive side chain with the plasma or by cleavage of the reactive side chain by the action of the plasma during deposition on the substrate. It is important that the reactive side chain can more readily be attacked by the plasma than can the chromophore. This has the advantage that, when a reactive side chain is used, on average a larger proportion of intact chromophores is deposited on the substrate.

In one embodiment of the method according to the invention, the chromophore of the UV absorber is selected from triazine, biphenyltriazine, benzotriazole, benzophenone, resorcinol, cyanoacrylate and derivatives thereof, as well as cinnamic acid derivatives. These chromophores impart particularly high OD340 values to the functional layer produced by the method according to the invention. Particularly preferably, the chromophore of the UV absorber is selected from benzotriazole, benzophenone, resorcinol, cyanoacrylate, derivatives thereof and cinnamic acid derivative. Surprisingly, these chromophores exhibit high stability in the plasma treatment.

Most particularly preferably, the chromophore to be used according to the invention is selected from benzotriazole and resorcinol. Of the chromophores that are more stable in the plasma, the benzotriazoles and resorcinols exhibit particularly high UV absorption in the end product.

As well as comprising the at least one reactive side chain, a chromophore within the meaning of the invention can comprise further substituents. These substituents can be selected from H, halogen, CN, SH, OH, —NH₂, COOH, C₁₋₁₀-alkyl, C₁₋₁₀-alkoxy, C₁₋₁₀-alkenyl, —N—C₁₋₁₀-alkyl and alkylaryl.

In one embodiment of the method according to the invention, the reactive side chain of the UV absorber comprises an ethylenically unsaturated double bond, an alkoxyalkylsilyl group or both structural elements. Surprisingly, it has been shown that ethylenically unsaturated double bonds and alkoxyalkylsilyl groups in the side chains lead to particularly high OD340 values of the deposited layer. This suggests that the unsaturated double bonds and the alkoxyalkylsilyl groups protect the chromophores of the UV absorbers particularly effectively during deposition in the plasma. Suitable functional structural units comprising at least one ethylenically unsaturated double bond in the side chain of the UV absorber and can be based inter alia on α,β-unsaturated carboxylic acid derivatives such as acrylates, methacrylates, maleates, fumarates, maleimides, acrylamides, or on compounds comprising vinyl ether, propenyl ether, allyl ether and dicyclopentadienyl units. Vinyl ethers, acrylates and methacrylates are preferred, and acrylates and methacrylates are particularly preferred.

Examples of UV absorbers comprising at least one chromophore and at least one of the above-described reactive side chains which can be used according to the invention are listed hereinbelow:
2-hydroxybenzophenones described in U.S. Pat. No. 5,041,313 and EP 0 570 165 B1
resorcinols described in U.S. Pat. No. 5,679,820, U.S. Pat. No. 5,391,795 and U.S. Pat. No. 5,981,073 (Ex. 5)
biphenyltriazines described in EP 2 447 236 and WO 1999/055471 A1
triazines described in DE 197 397 81, U.S. Pat. No. 6,500,887, U.S. Pat. No. 5,869,588 and U.S. Pat. No. 5,672,704
benzotriazoles described in EP 1 077 952, in particular 2-(2′-hydroxy-5′-methacryloxyethylphenyl)-2H-benzotriazole; commercial light stabiliser Tinuvin® R796)

In a preferred embodiment of the method according to the invention, the UV absorber is selected from the group of the 2-(2′-hydroxy-5′-(meth)acryloxyalkoxyalkylphenyl)-2H-benzotriazoles.

Surprisingly, it has been shown that the combination of chromophore and reactive side chain in this group of UV absorbers, relative to the amount of UV absorber used, has particularly high UV absorption after deposition in the plasma. Particular preference is given to 2-(2′-hydroxy-5′-methacryloxyethylphenyl)-2H-benzotriazole (Tinuvin® R796).

For evaporation of the UV absorber, it can be present, for example, in a heatable container, for example a boat. The container can be made of metal, for example, and be heated by a heating current.

Before evaporation of the UV absorber in the vacuum chamber, a pressure in the range of from 10⁻¹⁰ bar to 10⁻² bar, particularly preferably in the range of from 10⁻⁹ bar to 10⁻⁴ bar, in particular in the range of from 10⁻⁸ bar to 10⁻⁵ bar, is preferably established in the vacuum chamber.

The organic UV absorber is evaporated until preferably a pressure in the range of from 10⁻⁷ bar to 10⁻¹ bar is reached, particularly preferably in the range of from 10⁻⁶ bar to 10⁻³ bar, in particular in the range of from 10⁻⁵ bar to 10⁻⁴ bar.

Depending on the type of UV absorber and the pressure established in the vacuum chamber, the container containing the organic UV absorber is heated to a temperature which leads to evaporation of the organic UV absorber.

The distance between the boat containing the organic UV absorber and the plastics substrate is chosen in the range of from 1 cm to 1 m, particularly preferably in the range of from 2 cm to 20 cm, in particular in the range of from 5 cm to 15 cm.

Preferably, no additional initiators such as, for example, mono- or poly-ethylenic compounds are used.

Plasma

According to the invention, the gaseous organic UV absorber is deposited by a plasma. A plasma within the meaning of the invention is a gas whose constituents are “divided” partially or completely into ions and electrons. That is to say, a plasma contains free charge carriers. A low-pressure plasma is a plasma in which the pressure is considerably lower than atmospheric pressure. Low-pressure plasmas belong to the non-thermal plasmas, that is to say the individual constituents of the plasma (ions, electrons, neutral particles) are not in thermal equilibrium with one another. Typical industrial low-pressure plasmas are operated in the pressure range below 100 mbar, that is to say at pressures which are lower by a factor of 10 than normal air pressure. In the case of industrial low-pressure plasmas, electron temperatures of a few electron volts (several 10,000 K) are achieved by selective excitation of the electrons, while the temperature of the neutral gas is slightly above room temperature. As a result, even thermally sensitive materials such as plastics materials can be treated by means of low-pressure plasmas. The interaction of the plasma with the workpiece occurs by simple contacting.

Suitable methods within the meaning of this application for producing an industrial plasma are those which are ignited by means of electric discharge at a pressure that is reduced as compared with normal pressure of 1013 mbar, using a direct current, high-frequency or microwave excitation. These methods are known in the art by the name low-pressure or low-temperature plasma.

In the low-pressure plasma method, the workpiece to be treated is located in a vacuum chamber which can be evacuated by means of pumps.

This vacuum chamber includes at least one electrode when the plasma is excited by electrical excitation by means of direct current or by high-frequency fields. There can be used as the excitation frequency, for example: 13.56 MHz, 27.12 MHz or preferably 2.45 GHz. For the preferred case that the excitation takes place by means of microwave radiation, there could be, for example, at one point of the chamber wall a region which is permeable to microwave radiation and through which the microwave radiation is coupled into the chamber. Another preferred possibility consists in coupling the microwave power along a microwave-permeable tube, for example made of quartz glass. Such an arrangement is called Duo-Plasmaline (Muegge Electronic, Reichelsheim, Germany). These microwave sources are typically operated by two 2.45 GHz magnetrons. The plasma then burns along the tubes and can thus easily be extended over large workpieces.

According to the invention, the working pressure is more than 10⁻⁵ bar but below atmospheric pressure of 1.013 bar. This pressure range is advantageous because UV absorbers having a relatively low vapour pressure (relatively low molecular weight) can be used.

It has been found, surprisingly, that improved layer formation of the organic UV absorber is achieved in the pressure range above 10⁻⁵ bar. It was possible in particular to form layers having a high OD340 value. In addition, the layers deposited in that range exhibited good adhesion to the substrate. In a preferred embodiment, the working pressure is in the range of from 2×10⁻⁵ bar to 10⁻³ bar, in particular in the range of from 3×10⁻⁵ bar to 10⁻⁴ bar. In that pressure range, the UV absorber layers were achieved with particularly good optical values, in particular UV protection, clarity, haze, yellowness, and particularly good adhesion to the plastics substrate.

In a preferred embodiment of the invention, a microwave plasma is used to deposit the UV absorber.

Because gas molecules are very mobile and the electric discharge that generates the plasma fills the whole of the recipient almost uniformly, a plasma is very suitable for the uniform treatment of workpieces of complex shapes, for example with bores or undercuts. It is likewise possible, using a suitable holder, to treat flat shaped articles on both sides in one process step.

Suitable process gases for the deposition of the UV absorber in vapour form are argon, oxygen and nitrogen. Argon and oxygen are particularly preferred.

In a further embodiment of the method according to the invention, a precursor is introduced into the plasma zone during the deposition of the organic UV absorber and is deposited together with the organic UV absorber. As a result, the deposited UV absorber is present in a matrix on the plastics substrate formed at least in part of the precursor. The matrix is formed on the plastics substrate by plasma polymerisation. By incorporating the UV absorber into a matrix, improved adhesion of the UV absorber layer can be achieved, for example. In addition, the optical and mechanical properties of the layer can be improved.

The distance between the plasma source and the plastics substrate is chosen in the range of from 1 cm to 1 m, particularly preferably in the range of from 5 cm to 80 cm, in particular in the range of from 10 cm to 70 cm.

The distance to be chosen between the plasma source and the plastics substrate is dependent on the dimensions of the installation and the position of the UV absorber vapour source. The distance can be so adjusted that the plasma reaches the substrate. The distance is in particular so adjusted that sufficient excitation of the UV absorber for deposition on the plastics substrate is ensured. In one embodiment of the method according to the invention, the plasma source and the UV absorber source can be arranged spaced apart from one another at the same height, in such a manner that the direction of diffusion of the UV absorber vapour and the plasma flow direction are parallel to one another. In this embodiment, the plastics substrate is arranged on a movable holder in such a manner that a surface of the plastics substrate can be exposed either to the UV absorber gas stream or to the plasma. A surface of the plastics substrate is in this embodiment first exposed to the UV absorber vapour, gaseous UV absorber being deposited on the surface. The holder with the plastics substrate is subsequently moved from the UV absorber vapour source to the plasma source. The UV absorber that has remained on the surface of the plastics substrate is activated by the plasma and thereby forms a layer on the plastics substrate.

Plasma Polymerisation

Within the meaning of the invention, plasma polymerisation is used synonymously with plasma-enhanced vapour deposition (PECVD). Plasma polymerisation is defined, for example, in “G. Benz: Plasmapolymerisation: Überblick and Anwendung als Korrosions-und Zerkratzungsschutzschichten. VDI-Verlag GmbH Düsseldorf, 1989” or in “Vakuumbeschichtung Bd. 2-Verfahren, H. Frey, VDI-Verlag Düsseldorf 1995”.

Precursor compounds (precursors) in vapour form are first activated in the vacuum chamber by a plasma. The activation results in the formation of ionised molecules, and initial molecule fragments in the form of clusters or chains already form in the gas phase. The subsequent condensation of these fragments on the substrate surface then brings about polymerisation, under the effect of the substrate temperature and electron and ion bombardment, and thus the formation of a closed layer.

In a preferred embodiment, the UV absorber is deposited on the surface of the plastics substrate in a matrix which imparts scratch resistance.

Precursors which impart scratch resistance are, for example, siloxanes, which are introduced in vapour form into the vacuum chamber and are oxidised by means of an O₂ plasma to SiO₂, which is precipitated in the form of a glass-like scratch-resistant layer on the substrate. The components such as carbon and hydrogen which are also present thereby react to form carbon-containing gases and also water. The hardness of the layers can be adjusted by the concentration of siloxane to the oxygen gas. Low oxygen concentrations tend to lead to viscous layers, while high concentrations produce glass-like hard layers.

Examples of precursors which impart scratch resistance are hexamethyldisiloxane, octamethyltrisiloxane, decamethyltetrasiloxane, dodecamethylpentasiloxane, hexamethylcyclotrisiloxane, octamethylcyclotetrasiloxane, decamethylcyclopentasiloxane, dodecamethylcyclohexasiloxane, tetramethylcyclotetrasiloxane, tetraethoxysilane, tetramethyldisiloxane, trimethoxymethylsilane, dimethyldimethoxysilane, hexamethyldisilazane, triethoxyphenylsiloxane or vinylsilane.

Preference would be given to precursors from the group hexamethyldisiloxane, octamethylcyclotetrasiloxane, tetramethylcyclotetrasiloxane, tetraethoxysilane, tetramethyldisiloxane, trimethoxymethylsilane, dimethyldimethoxysilane, hexamethyldisilazane, triethoxyphenylsiloxane or vinylsilane.

Acetylene, benzene, hexafluorobenzene, styrene, ethylene, tetrafluoroethylene, cyclohexane, oxirane, acrylic acid, propionic acid, vinyl acetate, methyl acrylate, hexamethyldisilane, tetramethyldisilane and divinyltetramethyldisiloxane are further used.

In a further embodiment of the method according to the invention, before the organic UV absorber is applied, the plastics substrate is coated by means of PECVD with a layer imparting scratch resistance. It is thereby possible to impart to the plastics substrate both UV protection and scratch resistance. In addition, this first layer applied by PECVD can serve as an adhesion promoter for the organic UV absorber. Adhesion promoters are understood as being layers to which another layer adheres better than to the substrate to which the adhesion promoter layer has been applied.

According to a further embodiment of the method according to the invention, before the UV absorber is applied, an adhesion promoter layer is applied by means of plasma-enhanced vapour deposition. The adhesion-promoting layer can also be scratch-resistant.

In a further preferred embodiment of the method according to the invention, at least one further functional layer is applied to the UV absorber layer under low-pressure conditions by means of plasma-induced vapour deposition.

According to a preferred embodiment of the method according to the invention, the surface of the plastics substrate is subjected to plasma pretreatment before the UV-absorber-comprising functional layer is deposited. A plasma gas in contact with the surface of the plastics substrate is thereby ignited. A plasma pretreatment has a positive effect inter alia on the adhesion of the UV-absorber-comprising functional layer.

The duration of the plasma pretreatment is dependent inter alia on the stream of process gas. The duration can be in the range of from 1 minute to 10 minutes. Below 1 minute, no noticeable effect is achieved with the plasma pretreatment. Above 10 minutes, no further enhancement of the effect is achieved. Preferably, the surface of the plastics substrate is pretreated for a period in the range of from 2 minutes to 8 minutes, in particular in the range of from 2 minutes to 5 minutes.

Plastics Substrate

The plastics substrate to be coated according to the invention can be a thermoplastically proces sable material.

Thermoplastically processable plastics materials within the meaning of the invention are preferably polycarbonate, co-polycarbonate, polyester carbonate, polystyrene, styrene copolymers, aromatic polyesters such as polyethylene terephthalate (PET), PET-cyclohexanedimethanol copolymer (PETG), polyethylene naphthalate (PEN), polybutylene terephthalate (PBT), aliphatic polyolefins such as polypropylene or polyethylene, cyclic polyolefin, poly- or copoly-acrylates and poly- or copoly-methacrylate such as poly- or copoly-methyl methacrylates (such as PMMA) as well as copolymers with styrene such as transparent polystyrene-acrylonitrile (PSAN), thermoplastic polyurethanes, polymers based on cyclic olefins (e.g. TOPAS®, a commercial product from Ticona), polycarbonate blends with olefinic copolymers or graft polymers, such as, for example, styrene/acrylonitrile copolymers. The above-mentioned polymers can be used on their own or in mixtures.

Preference is given to polycarbonate, co-polycarbonate, polyester carbonate, aliphatic polyolefins such as polypropylene or polyethylene, cyclic polyolefin, PET or PETG, as well as poly- or copoly-acrylates and poly- or copoly-methacrylate such as poly- or copoly-methyl methacrylates, and also mixtures of the above-mentioned polymers.

Particular preference is given to polycarbonate, co-polycarbonate, polyester carbonate, PET or PETG, as well as poly- or copoly-acrylates and poly- or copoly-methacrylate, such as poly- or copoly-methyl methacrylates, and also mixtures of the above-mentioned polymers.

Most particularly, a polycarbonate and/or a co-polycarbonate is used as the plastics substrate. A blend system which comprises at least one polycarbonate or co-polycarbonate is also preferred.

According to the invention, the plastics substrates to which the organic UV absorber is applied can be precoated with any desired other layers.

Polycarbonates

Polycarbonates within the meaning of the invention are homopolycarbonates, copolycarbonates and polyester carbonates, as are described in EP 1 657 281 A.

The preparation of aromatic polycarbonates is carried out, for example, by reacting diphenols with carbonic acid halides, preferably phosgene, and/or with aromatic dicarboxylic acid dihalides, preferably benzenedicarboxylic acid dihalides, by the interfacial process, optionally using chain terminators, for example monophenols, and optionally using trifunctional or more than trifunctional branching agents, for example triphenols or tetraphenols. Preparation by a melt polymerisation process by reacting diphenols with, for example, diphenyl carbonate is likewise possible.

Diphenols for the preparation of the aromatic polycarbonates and/or aromatic polyester carbonates are preferably those of formula (I)

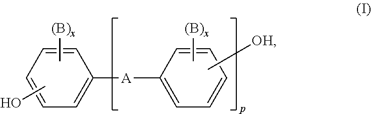

wherein

A denotes a single bond, C₁- to C₅-alkylene, C₂- to C₅-alkylidene, C₅- to C₆-cycloalkylidene, —O—, —SO—, —CO—, —5—, —SO₂—, C₆- to C₁₂-arylene, to which further aromatic rings optionally containing hetero atoms may be fused,
or a radical of formula (II) or (III)

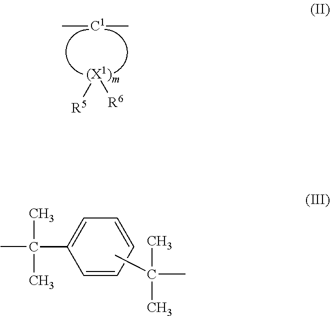

B denotes in each case C₁- to C₁₂-alkyl, preferably methyl, halogen, preferably chlorine and/or bromine,

x each independently of the other denotes 0, 1 or 2,

p are 0 or 1, and

R⁵ and R⁶ can be chosen individually for each X¹ and denote, independently of one another, hydrogen or C₁- to C₆-alkyl, preferably hydrogen, methyl or ethyl,

X¹ denotes carbon, and

m denotes an integer from 4 to 7, preferably 4 or 5, with the proviso that on at least one atom X¹, R⁵ and R⁶ are simultaneously alkyl.

Diphenols suitable for the preparation of the polycarbonates are, for example, hydroquinone, resorcinol, dihydroxydiphenyls, bis-(hydroxyphenyl)-alkanes, bis(hydroxyphenyl)-cycloalkanes, bis-(hydroxyphenyl) sulfides, bis-(hydroxyphenyl)ethers, bis-(hydroxyphenyl)ketones, bis-(hydroxyphenyl)-sulfones, bis-(hydroxyphenyl)sulfoxides, alpha-alpha′-bis-(hydroxyphenyl)-diisopropylbenzenes, phthalimidines derived from isatin or phenolphthalein derivatives, as well as compounds thereof alkylated and halogenated on the ring.

Preferred diphenols are 4,4′-dihydroxydiphenyl, 2,2-bis-(4-hydroxyphenyl)-propane, 2,4-bis-(4-hydroxyphenyl)-2-methylbutane, 1,1-bis-(4-hydroxyphenyl)-p-diisopropylbenzene, 2,2-bis-(3-methyl-4-hydroxyphenyl)-propane, 2,2-bis-(3-chloro-4-hydroxyphenyl)-propane, bis-(3,5-dimethyl-4-hydroxyphenyl)-methane, 2,2-bis-(3,5-dimethyl-4-hydroxy-phenyl)-propane, bis-(3,5-dimethyl-4-hydroxyphenyl)-sulfone, 2,4-bis-(3,5-dimethyl-4-hydroxyphenyl)-2-methylbutane, 1,1-bis-(3,5-dimethyl-4-hydroxyphenyl)-p-diisopropyl-benzene, 2,2-bis-(3,5-dichloro-4-hydroxyphenyl)-propane, 2,2-bis-(3,5-dibromo-4-hydroxyphenyl)-propane, 1,1-bis-(4-hydroxyphenyl)-3,3,5-trimethylcyclohexane, and 2-hydrocarbyl-3,3-bis(4-hydroxyaryl)phthalimidines, and the reaction product of N-phenylisatin and phenol.

Particularly preferred diphenols are 2,2-bis-(4-hydroxyphenyl)-propane, 2,2-bis-(3,5-dimethyl-4-hydroxyphenyl)-propane, 2,2-bis-(3 ,5-dichloro-4-hydroxyphenyl)-propane, 2,2-bis-(3,5-dibromo-4-hydroxyphenyl)-propane, 1,1-bis-(4-hydroxyphenyl)-cyclohexane and 1,1-bis-(4-hydroxyphenyl)-3,3,5-trimethylcyclohexane. In the case of the homopolycarbonates, only one diphenol is used; in the case of the co-polycarbonates, a plurality of diphenols is used. Suitable carbonic acid derivatives are, for example, phosgene or diphenyl carbonate.

Suitable chain terminators which can be used in the preparation of the polycarbonates are both monophenols and monocarboxylic acids. Suitable monophenols are phenol itself, alkylphenols such as cresols, p-tert-butylphenol, cumylphenol, p-n-octylphenol, p-isooctyl-phenol, p-n-nonylphenol and p-isononylphenol, halophenols such as p-chlorophenol, 2,4-dichlorophenol, p-bromophenol and 2,4,6-tribromophenol, 2,4,6-triiodophenol, p-iodophenol, and also mixtures thereof. Preferred chain terminators are phenol, cumylphenol and/or p-tert-butylphenol.

Particularly preferred polycarbonates within the context of the present invention are homopolycarbonates based on bisphenol A and copolycarbonates based on monomers selected from at least one of the group of bisphenol A, 1,1-bis-(4-hydroxyphenyl)-3,3,5-trimethylcyclohexane, 2-hydrocarbyl-3,3-bis(4-hydroxyaryl)phthalimidines and the reaction products of N-phenylisatin and phenol. The polycarbonates can in known manner be linear or branched. The proportion of co-monomers, based on bisphenol A, is generally up to 60 wt. %, preferably up to 50 wt. %, particularly preferably from 3 to 30 wt. %. Mixtures of homopolycarbonate and copolycarbonates can likewise be used.

Polycarbonates and co-polycarbonates comprising 2-hydrocarbyl-3,3-bis(4-hydroxyaryl)phthalimidines as monomers are known inter alia from EP 1 582 549 A1. Polycarbonates and co-polycarbonates comprising bisphenol monomers based on reaction products of N-phenylisatin and phenol are described, for example, in WO 2008/037364 A1.

Also suitable are polycarbonate-polysiloxane block cocondensates. The block cocondensates preferably comprise blocks of dimethylsiloxane. The preparation of polysiloxane-poolycarbonate block cocondensates is described, for example, in U.S. Pat. No. 3,189,662 A, U.S. Pat. No. 3 419 634 A and EP 0 122 535 A1. The block cocondensates preferably comprise from 1 wt. % to 50 wt. %, preferably from 2 wt. % to 20 wt. %, dimethylsiloxane.

The thermoplastic, aromatic polycarbonates have mean molecular weights (weight average Mw, measured by GPC (gel permeation chromatography with polycarbonate standard) of from 10,000 g/mol to 80,000 g/mol, preferably from 14,000 g/mol to 32,000 g/mol, particularly preferably from 18,000 g/mol to 32,000 g/mol. In the case of injection-moulded polycarbonate mouldings, the preferred mean molecular weight is from 20,000 g/mol to 29,000 g/mol. In the case of extruded polycarbonate mouldings, the preferred mean molecular weight is from 25,000 g/mol to 32,000 g/mol.

The thermoplastic plastics materials of the carrier layer can further comprise fillers. In the present invention, fillers have the purpose of lowering the thermal expansion coefficient of the polycarbonate and adjusting, preferably lowering, the permeability of gases and water vapour. Suitable fillers are glass beads, hollow glass beads, glass flakes, carbon blacks, graphite, carbon nanotubes, quartz, talc, mica, silicates, nitrides, wollastonite, as well as pyrogenic or precipitated silicas. the silicas having BET surface areas of at least 50 m²/g (in accordance with DIN 66131/2).

Preferred fibrous fillers are metal fibres, carbon fibres, plastics fibres, glass fibres or ground glass fibres, particular preference being given to glass fibres or ground glass fibres.

Preferred glass fibres are also those which are used in the form of endless fibres (rovings), long glass fibres and chopped glass fibres, which are produced from M-, E-, A-, S-, R- or C-glass, with E-, A- or C-glass being further preferred. The diameter of the fibres is preferably from 5 μm to 25 μm, more preferably from 6 μm to 20 μm, particularly preferably from 7 μm to 15 μm. Long glass fibres preferably have a length of from 5 μm to 50 mm, more preferably from 5 μm to 30 mm, yet more preferably from 6 μm to 15 mm, and particularly preferably from 7 μm to 12 mm; they are described, for example, in WO 2006/040087 A1. In the case of chopped glass fibres, preferably at least 70 wt. % of the glass fibres have a length of more than 60 μm. Further inorganic fillers are inorganic particles having a particle shape selected from the group comprising spherical, cubic, tabular, discus-shaped and plate-like geometries. Particularly suitable are inorganic fillers with a spherical or plate-like geometry, preferably in finely divided and/or porous form with a large outer and/or inner surface area. They are preferably thermally inert inorganic materials based in particular on nitrides such as boron nitride, oxides or mixed oxides such as cerium oxide, aluminium oxide, carbides such as tungsten carbide, silicon carbide or boron carbide, powdered quartz such as quartz flour, amorphous SiO₂, ground sand, glass particles such as glass powder, in particular glass beads, silicates or aluminosilicates, graphite, in particular highly pure synthetic graphite. Particular preference is given to quartz and talc, most preferably quartz (spherical particle shape). These fillers are characterised by a mean diameter d50% of from 0.1 μm to 10 μm, preferably from 0.2 μm to 8.0 μm, more preferably from 0.5 μm to 5 μm.

Silicates are characterised by a mean diameter d50 of from 2 μm to 10 μm, preferably from 2.5 μm to 8.0 μm, more preferably from 3 μm to 5 μm, and particularly preferably of 3 μm, an upper diameter d95% of accordingly from 6 μm to 34 μm, more preferably from 6.5 μm to 25.0 μm, yet more preferably from 7 μm to 15 μm, and particularly preferably of 10 μm being preferred. Preferably, the silicates have a specific BET surface area, determined by nitrogen adsorption in accordance with ISO 9277, of from 0.4 m²/g to 8.0 m²/g, more preferably from 2 m²/g to 6 m²/g, and particularly preferably from 4.4 m²/g to 5.0 m²/g. Further preferred silicates have only a maximum of 3 wt. % secondary constituents, whereby preferably the content of Al₂O₃<2.0 wt. %, Fe₂O₃<0.05 wt. %, (CaO+MgO)<0.1 wt. %, (Na₂O+K₂O)<0.1 wt. %, in each case based on the total weight of the silicate.

Further silicates use wollastonite or talc in the form of finely ground types having a mean particle diameter d50 of <10 μm, preferably <5 μm, particularly preferably <2 μm, most particularly preferably <1.5 μm. The particle size distribution is determined by air classification. The silicates can have a coating of organosilicon compounds, there preferably being used epoxysilane, methylsiloxane and methacrylsilane sizes. An epoxysilane size is particularly preferred.

The fillers can be added in an amount of up to 40 wt. %, based on the amount of polycarbonate. Preference is given to from 2.0 wt. % to 40.0 wt. %, particularly preferably from 3.0 wt. % to 35.0 wt. %.

Suitable blend partners for the thermoplastic plastics materials, in particular for polycarbonates, are graft polymers of vinyl monomers on graft bases such as diene rubbers or acrylate rubbers. Graft polymers B are preferably those of B.1 from 5 wt. % to 95 wt. %, preferably from 30 wt. % to 90 wt. %, of at least one vinyl monomer on B.2 from 95 wt. % to 5 wt. %, preferably from 70 wt. % to 10 wt. %, of one or more graft bases having glass transition temperatures <10° C., preferably <0° C., particularly preferably <−20° C. The graft base B.2 generally has a mean particle size (d50 value) of from 0.05 μm to 10 μm, preferably from 0.1 μm to 5 μm, particularly preferably from 0.2 μm to 1 μm. Monomers B.1 are preferably mixtures of B.1.1 from 50 to 99 parts by weight of vinyl aromatic compounds and/or vinyl aromatic compounds substituted on the ring (such as styrene, *-methylstyrene, p-methylstyrene, p-chlorostyrene) and/or methacrylic acid (C₁-C₈)-alkyl esters (such as methyl methacrylate, ethyl methacrylate), and

B.1.2 from 1 μm to 50 parts by weight of vinyl cyanides (unsaturated nitriles such as acrylonitrile and methacrylonitrile) and/or (meth)acrylic acid (C₁-C₈)-alkyl esters, such as methyl methacrylate, n-butyl acrylate, tert-butyl acrylate, and/or derivatives (such as anhydrides and imides) of unsaturated carboxylic acids, for example maleic anhydride and N-phenyl-maleimide. Preferred monomers B.1.1 are selected from at least one of the monomers styrene, * methylstyrene and methyl methacrylate, preferred monomers B.1.2 are selected from at least one of the monomers acrylonitrile, maleic anhydride and methyl methacrylate. Particularly preferred monomers are B.1.1 styrene and B.1.2 acrylonitrile.

Suitable graft bases B.2 for the graft polymers B are, for example, diene rubbers, EP(D)M rubbers, that is to say those based on ethylene/propylene and optionally diene, acrylate, polyurethane, silicone, chloroprene and ethylene/vinyl acetate rubbers. Preferred graft bases B.2 are diene rubbers, for example based on butadiene and isoprene, or mixtures of diene rubbers or copolymers of diene rubbers or mixtures thereof with further copolymerisable monomers (e.g. according to B.1.1 and B.1.2), with the proviso that the glass transition temperature of the graft base B.2 is below 10° C., preferably <0° C., particularly preferably <−10° C. Pure polybutadiene rubber is particularly preferred.

Particularly preferred polymers B are, for example, ABS polymers (emulsion, mass and suspension ABS), as are described, for example, in DE 2 035 390 A1 or in DE 2 248 242 A1 or in Ullmanns, Enzyklopadie der Technischen Chemie, Vol. 19 (1980), p. 280 ff. The gel content of the graft base B.2 is at least 30 wt. %, preferably at least 40 wt. % (measured in toluene). The graft copolymers B are prepared by radical polymerisation, for example by emulsion, suspension, solution or mass polymerisation, preferably by emulsion or mass polymerisation. Because, as is known, the graft monomers are not necessarily grafted completely onto the graft base in the graft reaction, graft polymers B are also understood as being products that are obtained by (co)polymerisation of the graft monomers in the presence of the graft base and that also form upon working up. The polymer compositions can optionally also comprise further conventional polymer additives, such as, for example, the antioxidants, heat stabilisers, demoulding agents, optical brighteners, UV absorbers and light-scattering agents described in EP 0 839 623 A1, WO 96 15102 A1, EP 0 500 496 A1 or “Plastics Additives Handbook”, Hans Zweifel, 5th Edition 2000, Hanser Verlag, Munich), in the amounts conventional for the thermoplastics in question.

Suitable UV stabilisers are benzotriazoles, triazines, benzophenones and/or arylated cyanoacrylates. Particularly suitable UV absorbers are hydroxy-benzotriazoles, such as 2-(3′,5′-bis-(1,1-dimethylbenzyl)-2′-hydroxy-phenyl)-benzotriazole (Tinuvin® 234, BASF SE, Ludwigshafen), 2-(2′-hydroxy-5′-(tert-octyl)-phenyl)-benzotriazole (Tinuvin® 329, BASF SE, Ludwigshafen), 2-(2′-hydroxy-3′-(2-butyl)-5′-(tert-butyl)-phenyl)-benzotriazole (Tinuvin® 350, BASF SE, Ludwigshafen), bis-(3-(2H-benztriazolyl)-2-hydroxy-5-tert-octyl)methane (Tinuvin® 360, BASF SE, Ludwigshafen), 2-(4,6-diphenyl-1,3,5-triazin-2-yl)-5-(hexyloxy)-phenol (Tinuvin® 1577, BASF SE, Ludwigshafen), and the benzophenones 2,4-dihydroxy-benzophenone (Chimasorb® 22, BASF SE, Ludwigshafen) and 2-hydroxy-4-(octyloxy)-benzophenone (Chimasorb® 81, BASF SE, Ludwigshafen), 2-propenoic acid, 2-cyano-3,3-diphenyl-2,2-bis[[(2-cyano-1-oxo-3,3-diphenyl-2-propenyl)-oxyl]-methyl]-1,3-propanediyl ester (9Cl) (Uvinul® 3030, BASF SE, Ludwigshafen), 2-[2-hydroxy-4-(2-ethylhexyl)oxyl]phenyl-4,6-di(4-phenyl)phenyl-1,3,5-triazine (Tinuvin® 1600, BASF SE, Ludwigshafen) or tetra-ethyl-2,2′-(1,4-phenylene-dimethylidene)-bismalonate (Hostavin® B-Cap, Clariant AG). The composition of the thermoplastic plastics materials can comprise UV absorber conventionally in an amount of from 0 to 10 wt. %, preferably from 0.001 wt. % to 7.000 wt. %, particularly preferably from 0.001 wt. % to 5.000 wt. %, based on the total composition. The preparation of the compositions of the thermoplastic plastics materials is carried out by conventional incorporation methods by combining, mixing and homogenising the individual constituents, the homogenisation in particular preferably taking place in the melt under the action of shear forces. Combining and mixing optionally take place prior to the melt homogenisation using powder premixtures.

The thermoplastically processable plastics material can be processed to moulded articles in the form of films or sheets. The film or sheet can be of single- or multi-layer form and can consist of different or the same thermoplastics, for example polycarbonate/PMMA, polycarbonate/PVDF or polycarbonate/PTFE or also polycarbonate/polycarbonate.

The thermoplastically processable plastics material can be shaped, for example, by injection moulding or extrusion. By using one or more side extruders and a multichannel die or optionally suitable melt adapters upstream of a sheet die, thermoplastic melts of different compositions can be laid on top of one another and multilayer sheets or films can thus be produced (for coextrusion see, for example, EP-A 0 110 221, EP-A 0 110 238 and EP-A 0 716 919, for details of the adapter and die method see Johannaber/Ast: “Kunststoff-Maschinenführer”, Hanser Verlag, 2000 and in Gesellschaft Kunststofftechnik: “Koextrudierte Folien und Platten: Zukunftsperspektiven, Anforderungen, Anlagen und Herstellung, Qualitätssicherung”, VDI-Verlag, 1990). Polycarbonates and poly(meth)acrylates are preferably used for coextrusion. Polycarbonates are particularly preferably used.

The film can be shaped and back injection moulded with a further thermoplastic from the above-mentioned thermoplastics (Film Insert Moulding (FIM)). Sheets can be thermoformed or processed by means of drape forming or bent while cold. Shaping by injection moulding processes is also possible. These processes are known to the person skilled in the art. The thickness of the film or sheet must be such that sufficient rigidity is ensured in the component. In the case of a film, it can be strengthened by back injection moulding in order to ensure sufficient rigidity.

The total thickness of the moulded article produced from the thermoplastically processable plastics material, that is to say including a possible back injection moulding or coextruded layers, is generally from 0.1 mm to 15 mm. Preferably, the thickness of the moulded article is from 0.8 mm to 10 mm. In particular, the indicated thickness relates to the total thickness of the moulded article when polycarbonate is used as the material of the moulded article, including a possible back injection moulding or coextruded layers.

An exemplary embodiment of the method according to the invention will be described hereinbelow with reference to the figures.

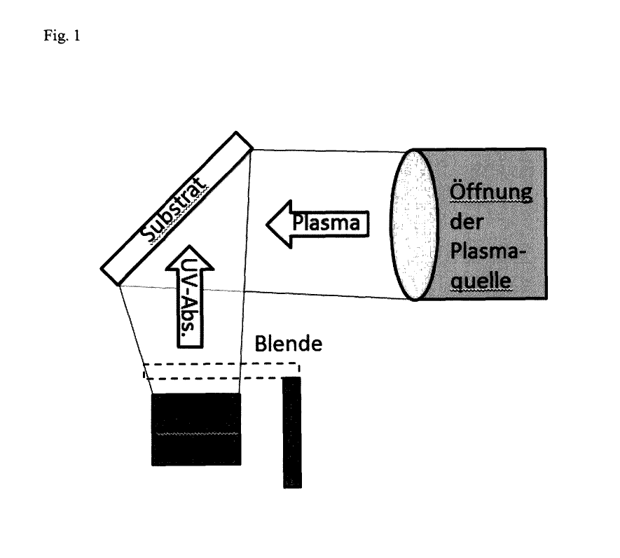

FIG. 1 shows a schematic representation of the method sequence according to the invention.

The vacuum chamber is evacuated to p<5·10⁻⁸ bar prior to the coating. The substrate is introduced as shown in FIG. 1 at a 45° angle to each of the vapour source and the plasma source. The vacuum chamber contains a boat which is made of molybdenum and has the following dimensions: W×H×D=100×25×38 mm. The boat further has a perforated cover from Umicore. The boat is covered by a pivoting shutter and is filled with about 2 g of UV absorber powder, so that the bottom is amply covered. After the coating operation, the boat is cleaned with solvent in an ultrasound bath and filled with fresh UV absorber for the next process.

The distance of the substrate middle to the boat is from 8 cm to 10 cm. In order to permit a controlled vapour deposition process, the UV absorber is melted slowly under a high vacuum. The operation is so controlled that the rate monitor (quartz crystal microbalance—not shown in the drawing) projecting laterally beneath the shutter does not display a rate. The vapour deposition current is upregulated slowly from I=30 A stepwise to about 80 A to 90 A. The operation takes place with the shutter closed. After this step, the high vacuum is let off and the vacuum chamber is evacuated only by means of a Roots pump. The plasma source used is a DuoPlasmaline from Muegge Electronic GmbH (Reichelsheim), which was mounted on a 250 mm flange. The distance from the substrate to the plasma source is approximately 30 cm.

For the deposition of layers of HMDSO (hexamethyldisiloxane from Aldrich), the HMDSO is evaporated by means of a CEM liquid metering system from Bronkhorst and introduced into the vacuum chamber through the DuoPlasmaline. After contact with the plasma, the deposition and polymerisation of the excited precursor on the substrate take place.

The examples which follow serve to explain the invention in greater detail.

### EXAMPLES

### Example 1

### Sheet Production

For the coating tests, injection moulded rectangular sheets of optical grade of dimensions 150×105×3.2 mm with a side gate were prepared using Makrolon® M2808 (linear bisphenol A polycarbonate from Bayer AG, Leverkusen with a melt flow index (MFR) according to ISO 1133 of 10 g/10 min at 300° C. and a 1.2 kg load). The melt temperature was from 300 to 330° C. and the tool temperature was 100° C. Prior to processing, the granulate in question was dried for 5 hours at 120° C. in a vacuum drying cabinet.

The following process parameters apply for Examples 2 to 5 and Comparative Examples 1 to 3.

| | |
|---|---|
| Plastics substrate | Polycarbonate |
| | sheet from Example 1 |
| Shortest distance vapour source to substrate | 8 cm to 10 cm |
| Shortest distance plasma source to substrate | 30 cm |
| Angle vapour source substrate | 45° |
| Angle plasma source substrate | 45° |
| Plasma source | Muegge Electronic GmbH |
| | (Reichelsheim) |
| Vapour source | Molybdenum boat |
| Dimensions of the vapour source | 100 × 25 × 38 mm |
| Amount of UV absorber | about 2 g |

### Comparative Example 1

The polycarbonate sheet was introduced into the vacuum chamber and the chamber was evacuated to p<5×10⁻⁸ bar. The boat was filled with Tinuvin® R796 (BASF), and the UV absorber was melted slowly under a high vacuum. The procedure is so controlled that the rate monitor (quartz crystal microbalance—not shown in the drawing) projecting laterally beneath the shutter does not display a rate. The vapour deposition current is upregulated slowly from I=30 A stepwise to approximately 80 A to 90 A. The operation takes place with the shutter closed. Operation was then switched to the prevacuum pumping unit.

During the switchover phase, the heating current was maintained at about 50 A so that the UV absorber does not cool. Then, with the aid of Ar gas, the pressure was adjusted to p=7×10⁻⁵ bar and then the UV absorber was vapour deposited. To that end, the heating current (typically 90 A) was so adjusted that the uncalibrated rate monitor showed a constant rate of about 4 Å/s. Using these process settings, samples were produced with different vapour deposition times.

### Comparative Example 2

The polycarbonate sheet was introduced into the vacuum chamber, and the vacuum chamber was evacuated to p<5×10⁻⁸ bar. Operation was then switched to the prevacuum pumping unit, and the process took place in 4 steps:
1. Plasma pretreatment: The substrate was exposed to the plasma for 4 minutes with an Ar plasma at p=10⁻⁴ bar and a power of 2000 W pulsed (tₒₙ:t_{off}=5 ms:5 ms).
2. Adhesion promoter layer: A layer consisting of hexamethyldisiloxane (HMDSO) was deposited as the adhesion promoter layer. The layer was deposited in the course of 3 minutes at a power of 2000 W pulsed (tₒₙ:t_{off}=5 ms:5 ms) and at a pressure of p=7×10⁻⁵ bar. The flow of HMDSO was 24 g/h and that of Ar was 0.05 l/min.
3. UV absorber layer: The UV absorber Tinuvin® 1577 (BASF) was vapour deposited during a period of about 25 minutes at a pressure of p=7×10⁻⁵ bar. In parallel, a plasma of HMDSO/O₂ and Ar burned. The power was 2000 W pulsed (tₒₙ:t_{off}=5 ms:5 ms) and the flows were Ar=0.05 1 l/min, O₂=0.25 l/min and HMDSO=24 g/h. The vapour deposition current was about 75 A and the vapour deposition process was conducted in such a manner that the rate remained as constant as possible.
4. Top layer: The top layer was deposited with the same parameters as the adhesion promoter layer.

### Comparative Example 3

The polycarbonate sheet was introduced into the vacuum chamber, and the vacuum chamber was evacuated to the base pressure of p<5·10⁻⁸ bar. In order to minimise the influence of the plasma on the UV absorber during the vapour deposition and nevertheless incorporate the UV absorber into a matrix, the UV absorber was vapour deposited alternately with the HMDSO.

The following process steps were carried out in succession:
1. Plasma pretreatment: The substrate was exposed to the plasma for 4 minutes with an Ar plasma at p=10⁻⁴ bar and a power of 2000 W pulsed (tₒₙ:t_{off}=5 ms:5 ms).
2. Adhesion promoter layer: A layer consisting of hexamethyldisiloxane (HMDSO) was deposited as the adhesion promoter layer. The layer was deposited in the course of 3 minutes at a power of 2000 W pulsed (tₒₙ:t_{off}=5 ms:5 ms) and at a pressure of p=7×10⁻⁵ bar. The flow of HMDSO was 24 g/h and that of Ar was 0.05 l/min.
3. UV absorber layer: The UV absorber Tinuvin® 1577 (BASF) without a reactive side chain was vapour deposited for about 20 seconds at a pressure of p=7×10⁻⁵ bar. In parallel, an Ar plasma burned. The power was 1000 W pulsed (tₒₙ:t_{off}=5 ms:5 ms) and the argon gas flow was Ar=0.6 l/min. The vapour deposition current was about 75 A and the vapour deposition process was conducted in such a manner that the rate remained as constant as possible.
4. Intermediate layer: An HMDSO layer was applied for 2 minutes as an intermediate layer. The power was 2000 W pulsed (tₒₙ:t_{off}=5 ms:5 ms) and the flows were O₂=0.25 l/min and HMDSO=24 g/h and the pressure was p=7×10⁻⁵ bar.
5. UV absorber layer: Repetition of method step 3
6. Intermediate layer: Repetition of method step 4
7. UV absorber layer: Repetition of method step 3
8. Intermediate layer: Repetition of method step 4
9. UV absorber layer: Repetition of method step 3
10. Intermediate layer: Repetition of method step 4
11. UV absorber layer: Repetition of method step 3
12. Intermediate layer: Repetition of method step 4
13. UV absorber layer: Repetition of method step 3
14. Top layer: The top layer was deposited with the same parameters as the adhesion promoter layer.

In order to determine the quality of the plastics substrates coated according to Comparative Examples 1 to 3, the parameters OD340, transmission (T_{y}), haze and yellowness (YI) were determined. The measurement results and the visual impression of the individual layers produced according to Comparative Examples 1 to 3 are summarised in Table 1.

TABLE 1

| | Vapour | | | | | |
|---|---|---|---|---|---|---|
| Comparative | deposition | | Haze | | Ty | |
| examples | time (s) | OD340 | (%) | YI | (%) | Layer quality |
| 1A | 5 | 0.43 | 43.4 | 3.05 | 86 | milky, spotted, |
| | | | | | | porous |
| 1B | 15 | 0.44 | 21 | 3.33 | 87 | milky, spotted, |
| | | | | | | porous |
| 1C | 30 | 0.56 | 31 | 2.68 | 87 | milky, spotted, |
| | | | | | | porous |
| 1D | 60 | 1.04 | 52.4 | 3.57 | 93 | milky, spotted, |
| | | | | | | porous |
| 1E | 90 | 1.14 | 59.5 | 4.12 | 79 | milky, spotted, |
| | | | | | | porous |
| 1F | 120 | 1.25 | 61.8 | 4.42 | 80 | milky, spotted, |
| | | | | | | porous |
| 2 | 1200 | 2.45 | 0.86 | 22.5 | 88 | yellowish- |
| | | | | | | brown, |
| | | | | | | homogeneous |
| 3 | 6 × 20 | 2.33 | 26.6 | 8.8 | 87 | yellow, |
| | | | | | | cloudy, |
| | | | | | | homogeneous |

### Example 2

The polycarbonate sheet was introduced into the vacuum chamber, and the vacuum chamber was evacuated to the base pressure of p<5·10⁻⁸ bar. The boat was filled with Tinuvin R796 (BASF) and the UV absorber was melted as described in Comparative Example 1, and then operation was switched to the prevacuum pump unit. During the switchover phase, the heating current was maintained at about 50 A so that the UV absorber does not cool. Then, with the aid of Ar gas, the pressure was adjusted to p=7×10⁻⁵ bar and then the plasma was ignited. The power was 2000 W pulsed (tₒₙ:t_{off}=5 ms:5 ms) and the UV absorber was vapour deposited at the same time as the plasma. To that end, the heating current (typically 90 A) was so adjusted that the uncalibrated rate monitor showed a constant rate of about 10 Å/s.

### Example 3

Coating was again carried out on the substrate of Example 1, and evacuation was carried out to the base pressure of p<5×10⁻⁸ bar. The boat was filled with Tinuvin® R796 (BASF).

The following process steps were carried out in succession:
1. Plasma pretreatment: The substrate was exposed to the plasma for 3 minutes with an Ar plasma at p=10⁻⁴ bar and a power of 2000 W pulsed (tₒₙ:t_{off}=5 ms:5 ms).
2. Adhesion promoter layer: A layer consisting of hexamethyldisiloxane (HMDSO) was deposited as the adhesion promoter layer. The layer was deposited in the course of 3 minutes at a power of 2000 W pulsed (tₒₙ:t_{off}=5 ms:5 ms) and at a pressure of p=7×10⁻⁵ bar. The flow of HMDSO was 24 g/h and that of Ar was 0.05 l/min.
3. Evacuation to p<5×10⁻⁸ bar.
4. Melting of the UV absorber as described above.
5. UV absorber deposition: With Ar gas, the pressure was adjusted to p=7·10⁻⁵ bar and then the plasma was ignited. The power was 2000 W pulsed (tₒₙ:t_{off}=5 ms:5 ms) and the UV absorber was vapour deposited at the same time as the plasma. To that end, the heating current (typically 90 A) was so adjusted that the uncalibrated rate monitor showed a constant rate of about 10 Å/s. The vapour deposition time was varied (see table).
6. Top layer of HMDSO: The layer was deposited analogously to the adhesion promoter layer.

Example 4

The polycarbonate sheet was introduced into the vacuum chamber, and the vacuum chamber was evacuated to the base pressure of p<5·10⁻⁸ bar. The boat was filled with Tinuvin® R796 (BASF).

The following process steps were carried out in succession:
1. Plasma pretreatment: The substrate was exposed to the plasma for 3 minutes with an Ar plasma at p=10⁻⁴ bar and a power of 2000 W pulsed (tₒₙ:t_{off}=5 ms:5 ms).
2. Adhesion promoter layer: A layer consisting of hexamethyldisiloxane (HMDSO) was deposited as the adhesion promoter layer. The layer was deposited in the course of 3 minutes at a power of 2000 W pulsed (tₒₙ:t_{off}=5 ms:5 ms) and at a pressure of p=7×10⁻⁵ bar. The flow of HMDSO was 24 g/h and that of Ar was 0.05 l/min.
3. Evacuation to p<5×10⁻⁸ bar.
4. Melting of the UV absorber as described in Comparative Example 1.
5. UV absorber deposition: With Ar gas, the pressure was adjusted to p=7×10⁻⁵ bar and then the plasma was ignited. The Ar flow was 0.47 l/min. The power was 2000 W pulsed (tₒₙ:t_{off}=5 ms:5 ms) and the UV absorber was vapour deposited at the same time as the plasma. To that end, the heating current (typically 90 A) was so adjusted that the uncalibrated rate monitor showed a constant rate of about 10 Å/s.
6. Top layer of HMDSO: The layer was deposited analogously to the adhesion promoter layer.

In order to determine the quality of the plastics substrates coated according to Examples 2 to 5, the parameters OD340, transmission (T_{y}), haze and yellowness (YI) were determined. The measurement results and the visual impression of the individual layers produced according to Examples 2 to 5 are summarised in Table 2.

TABLE 2

| Example | | | | | | |
|---|---|---|---|---|---|---|
| (according | Vapour | | | | | |
| to the | deposition | | Haze | | Ty | |
| invention) | time (s) | OD340 | (%) | YI | (%) | Layer quality |
| 2 | 30 | 2.3 | 0.48 | 2.9 | 89 | Clear, |
| | | | | | | transparent, |
| | | | | | | colourless, |
| | | | | | | homogeneous |
| 3A | 30 | 2.02 | 0.34 | 3.7 | 92 | Clear, |
| | | | | | | transparent, |
| | | | | | | colourless, |
| | | | | | | homogeneous |
| 3B | 60 | 2.25 | 0.48 | 2.9 | 92 | Clear, |
| | | | | | | transparent, |
| | | | | | | colourless, |
| | | | | | | homogeneous |
| 4 | 60 | 2.20 | 0.50 | 2.23 | 92 | Clear, |
| | | | | | | transparent, |
| | | | | | | colourless, |
| | | | | | | homogeneous |

The examples according to the invention show that, in order to achieve a preferred optical density at 340 nm (OD340) while at the same achieving low yellowness and haze, the UV absorber must have a side chain activated by a plasma. By contrast, the deposition of UV absorbers without a reactive side group with the action of plasma, and the deposition of UV absorber with a reactive side chain without the action of plasma, as demonstrated by the comparative examples, lead to cloudy and yellow layers without UV-protective action.

1. Method for coating a plastics substrate with a functional layer, wherein
(a) in a vacuum chamber, evaporating an organic UV absorber which comprises at least one chromophore and at least one reactive side chain and
(b) bringing said organic UV absorber into contact with at least one surface of the plastics substrate and excited with a plasma, or
(c) exciting said organic UV absorber with a plasma and then bringing said organic UV absorber into contact with at least one surface of the plastics substrate,
whereby a functional layer comprising the UV absorber is formed on the surface of the plastics substrate, the plasma being ignited at a pressure of greater than 10⁻⁵ bar and less than 1.013 bar.

2. Method according to claim 1, wherein the plasma is ignited at a pressure in the range of from 2 x 10⁻⁵ bar to 10⁻³ bar, optionally in the range of from 3×10⁻⁵ bar to 10⁻⁴ bar.

3. Method according to claim 1, wherein a chromophore of the organic UV absorber is selected from benzotriazole, benzophenone, resorcinol, cyanoacrylate and derivatives thereof and cinnamic acid derivatives, optionally from benzotriazole and resorcinol.

4. Method according to claim 1, wherein the reactive side chain of the organic UV absorber comprises an ethylenically unsaturated double bond and/or an alkoxyalkylsilyl group, optionally an acrylate and/or a methacrylate.

5. Method according to claim 1, wherein the UV absorber is selected from the group of the 2-(2′-hydroxy-5′-(meth)acryloxyalkoxyalkyl-phenyl)-2H-benzotriazoles, optionally 2-(2′-hydroxy-5′-methacryloxyethylphenyl)-2H-benzotriazole (Tinuvin® R796).

6. Method according to claim 1, wherein, before a first layer is deposited, a surface(s) of the plastics substrate is/are subjected to a plasma pretreatment and/or an adhesion-promoting layer is applied.

7. Method according to claim 1, wherein, during deposition of the organic UV absorber, a precursor is introduced into a plasma zone and is deposited together with the organic UV absorber, whereby the UV absorber is present in a matrix on the plastics substrate formed at least in part from the precursor.

8. Method according to claim 7, wherein the precursor is selected independently from hexamethyldisiloxane, octamethyltrisiloxane, decamethyltetrasiloxane, dodecamethylpentasiloxane, hexamethylcyclotrisiloxane, octamethylcyclotetrasiloxane, decamethylcyclopentasiloxane, dodecamethylcyclohexasiloxane, tetramethylcyclotetrasiloxane, tetraethoxysilane, tetramethyldisiloxane, trimethoxymethylsilane, dimethyldimethoxysilane, hexamethyldisilazane, triethoxyphenylsiloxane or vinylsilane, optionally from hexamethyldisiloxane, octamethylcyclotetrasiloxane, tetramethylcyclotetrasiloxane, tetraethoxysilane, tetramethyldisiloxane, trimethoxymethylsilane, dimethyldimethoxysilane, hexamethyldisilazane, triethoxyphenylsiloxane or vinylsilane.

9. Method according to claim 1, wherein at least one further functional layer is applied under low pressure conditions by plasma-induced vapour deposition.

10. Method according to claim 1, wherein the plastics substrate is a thermoplastically processable material, optionally polycarbonate, co-polycarbonate, polyester carbonate, PET or PETG, as well as poly- or copoly-acrylates and poly- or copoly-methacrylate such as poly- or copoly-methyl methacrylates, and also mixtures thereof.

11. Method according to claim 6, wherein the duration of the plasma pretreatment is in the range of from 1 minute to 10 minutes, optionally in the range of from 2 minutes to 8 minutes, optionally in the range of from 2 minutes to 5 minutes.

12. Method according to claim 1, wherein the plasma which is brought into contact with the organic UV absorber is a microwave plasma.

13. Coated plastics substrate comprising at least one UV-protective layer and optionally at least one scratch-resistant layer, wherein said subtrate has been produced by a method according to claim 1.

14. A coated plastics substrate according to claim 13 capable of being used as a casing for electronic devices, window profiles, headlamp diffusers, bodywork elements, machine covers, vehicle windows and architectural glazing.

15. An organic UV absorber having at least one chromophore and at least one reactive side chain capable of being used for coating plastics substrates, wherein the organic UV absorber is evaporated and brought into contact with a plasma.
